(19) United States
(12) Patent Application Publication
(10) Pub. No.: US 2015/0297623 A1
(43) Pub. Date: Oct. 22, 2015

(54) METHODS FOR TREATMENT OF PRIMARY CANCER AND CANCER METASTASIS

(71) Applicant: THE BOARD OF REGENTS OF THE UNIVERSITY OF TEXAS SYSTEM, Austin, TX (US)

(72) Inventors: Jean X. JIANG, San Antonio, TX (US); Jade Zifei ZHOU, San Antonio, TX (US)

(21) Appl. No.: 14/440,803

(22) PCT Filed: Nov. 6, 2013
(86) PCT No.: PCT/US13/68610

§ 371 Date: May 5, 2015

Related U.S. Application Data
(60) Provisional application No. 61/722,808, filed on Nov. 6, 2012.

(51) Int. Cl. A61K 31/7076; A61K 45/06; A61K 31/52; A61K 9/00; A61K 31/522

(52) U.S. Cl. CPC A61K 31/7076; A61K 9/0019; A61K 31/522; A61K 31/52; A61K 45/06

(57) ABSTRACT

Embodiments of the invention are directed to administering a therapeutically effective amount of a purinergic P2 receptor agonist alone or in combination with adenosine receptor antagonist and/or other anti-cancer therapies for the treatment of cancer. Agonist for the P2 receptors include non-hydrolysable ATP analogs. In particular aspects the cancer is a metastatic cancer, such as a bone metastasis.

This application claims priority to U.S. Provisional Patent Ser. No. 61/722,808 filed Nov. 6, 2012, which is incorporated herein by reference in its entirety.

### BACKGROUND

The bone is the most common site of metastasis in patients with advanced cancers including breast and prostate cancers (Jin et al. (2011) Int. J. Cancer 128, 2545-2561; Kohno, (2008) Int. J. Clin. Oncol. 13, 18-23). Bone metastases are major, potentially fatal complications in patients with advanced cancers. Almost all patients with skeletal metastases have significantly decreased quality of life due to intense pain, pathological fractures, spinal cord compression, and metabolic complications (Welch et al. (2003) J. Musculoskelet. Neuronal Interact. 3, 30-38). In fact, post-mortem studies have shown that over 70% of breast cancer patients exhibited skeletal metastases, and only 20% of these patients are still alive five years after the discovery of the metastases (Roodman (2004) N. Engl. J. Med. 350, 1655-1664; Welch et al. (2003) J. Musculoskelet. Neuronal Interact. 3, 30-38). The high affinity that cancer has for bone is explained by the “seed-and-soil hypothesis”, which was proposed over a century ago (Paget (1889) Lancet 1, 571-573). It reveals that bone tissues are preferred sites of cancer metastasis due to their microenvironment, which provides a fertile setting in which tumor cells can grow. Many features, such as increased blood flow as well as the release of growth factors from cells in the bone matrix, account for the frequency of bone metastases (van der Pluijm et al. (2001) J. Bone Miner. Res. 16, 1077-1091). Thus far, the critical factors and mechanisms responsible for bone metastases are largely unknown.

Bisphosphonate drugs are used to treat bone cancer metastasis and result in decreased tumor growth, reduced bone destruction, and reduced pain (Brown and Guise (2007) Cur. Osteopor. Rep. 5, 120-127). Unfortunately, bisphosphonate therapy is associated with serious adverse side effects, which include atrial fibrillation; arthralgia and osteonecrosis of the jaw; and ophthalmic, dermatologic and renal complications; as well as medication-induced fractures (Junquera et al. (2009) Am. J. Otolaryngol. 30, 390-395; Truong et al. (2010) J. Am. Acad. Dermatol. 62, 672-676). Despite advances in the diagnosis and treatment of bone metastasis from solid tumors, the mechanism of how bisphosphonate treatment inhibits bone metastasis at the molecular level remains to be established. It has been reported that alendronate (AD), a bisphosphonate drug, induces the opening of hemichannels, a channel permeable to small molecules (Mr<1 kDa) in osteocytes (Plotkin et al. (2002) J. Biol. Chem. 277, 8648-8657). In addition, hemichannels in osteocytes permit the release of ATP in response to mechanical loading (Genetos et al. (2007) J. Cell. Physiol. 212, 207-214). However, it is unknown whether the ATP release from osteocytes is responsible for the inhibitory effect of bisphosphonates on bone metastasis.

Previous studies point to the possibility that ATP through its binding to P2 purinergic receptors exhibits an anti-cancer effect (White and Burnstock (2006) Trends Pharmacol. Sci. 27, 211-217). Several studies have established the anti-neoplastic activity of ATP to inhibit the growth of several cell lines, including prostate cancer cells, colon adenocarcinoma cells, melanoma cells, and bladder cancer cells (Rapaport et al. (1983) Cancer Res. 43, 4402-4406; Shabbir and Burnstock (2009) Int. J. Urol. 16, 143-150; White and Burnstock (2006) Trends Pharmacol. Sci. 27, 211-217). The activation of purinergic signaling is also reported to inhibit proliferation and migration of human acute myeloblastic leukemia cells in immune-deficient mice (Salvestrini et al. (2012) Blood 119, 217-226). Additionally, in vivo studies show that daily injections of ATP significantly inhibit tumor growth, prolong survival time, and inhibit weight loss in mice (Rapaport (1988) Eur. J. Cancer Clin. Oncol. 24, 1491-1497). However, several studies also suggest adverse effects of ATP including increased tumor growth and migration.

There remains a need for additional therapies for treating cancer and in particular bone metastases.

### SUMMARY

The inventors demonstrate that ATP released from bone osteocytes inhibits the migration of cancer cells. In contrast to ATP, adenosine—a metabolite of ATP—promoted breast cancer cell migration. Adenosine stimulated breast cancer cell migration was attenuated by an adenosine receptor antagonist. These results suggest that adenosine nucleotides released from osteocytes impacts migration and growth of tumor cells, and is important in bone metastasis. Certain embodiments are directed to administration of purinergic P2 receptor agonist to treat cancer and reduce metastasis.

Purinergic P2 receptors are distinct from the P1 receptor and refers to receptors that bind to and are activated by adenosine-5′-triphosphate (ATP) or analogs thereof. P2X receptors are ATP activated channels that allow the passage of ions across cell membranes, whereas P2Y receptors are ATP activated G-protein coupled receptors (GPCR) that initiate intracellular signaling. Agonist for the P2 receptors include non-hydrolysable ATP analogs.

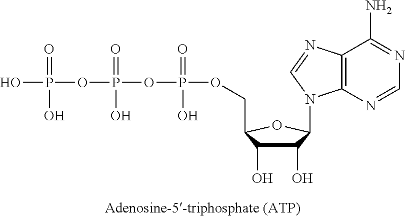

The term non-hydrolysable ATP analog refers to an ATP analog that is not effectively hydrolyzed by ATPase, i.e., the analog is hydrolyzed, if at all, at a rate that is less than 5, 1, or 0.1% of the rate of ATP hydrolysis by ATPase.

In certain aspects, non-hydrolysable ATP analogs include, but are not limited to adenosine 5′-[α-thio]triphosphate (ATPaS); alpha,beta-methylene-adenosine-5′-diphosphate (ApCpp); beta,gamma-methylene-ATP (AppCp); adenosine 5′[γ-thio]triphosphate (ATPγS); adenylyl imidodiphosphate (AMP-PNP); N⁶-diethyl-beta,gamma-dibromomethylene-ATP; 2-methylthio-ATP (APM); alpha,beta-methylene-ATP; beta,gamma-methylene-ATP; di-adenosine pentaphosphate (Ap5A); 1,N⁶-ethenoadenosine triphosphate; adenosine 1-oxide triphosphate; 2′,3′-O-(benzoyl-4-benzoyl)-ATP (BzATP); and 2′,3′-O-(2,4,6-trinitrophenyl)-ATP (TNP-ATP), the various structures of which can be found in the PubChem database on the world wide web at ncbi.nlm.nih.gov/pccompound (non-hydrolysable analogs can be purchased, for example, from Jena Biosciences, Jena, Germany; Sigma-Aldrich, St. Louis, Mo., USA).

Given that adenosine exposure can promote cancer cell growth and migration, and adenosine is produced by the metabolism of ATP, embodiments of the invention are directed to administering non-hydrolysable ATP analogs alone or in combination with adenosine receptor antagonist and/or other anti-cancer therapies for the treatment of cancer. Other embodiments are directed to treating cancer by administering adenosine receptor antagonist alone or in combination with non-hydrolysable ATP analogs and/or other anti-cancer therapies.

The adenosine receptors (or P1 receptors) are a class of purinergic receptors with adenosine as an endogenous ligand.

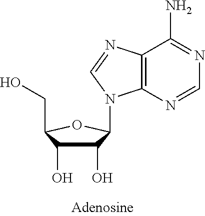

Certain embodiments include adenosine receptor antagonist. In certain aspects the adenosine receptor antagonist include antagonist specific for adenosine receptor A2B. Adenosine receptor antagonist include, but are not limited to N-(4-Cyanophenyl)-2-[4-(2,3,6,7-tetrahydro-2,6-dioxo-1,3-dipropyl-1H-purin-8-yl)phenoxy]-acetamide (MRS 1754, CAS no. 264622-58-4); N-(4-Acetylphenyl)-2-[4-(2,3,6,7-tetrahydro-2,6-dioxo-1,3-dipropyl-1H-purin-8-yl)phenoxy]acetamide (MRS 1706, CAS No. 264622-53-9); 8-[4-[4-(4-Chlorobenzyl)piperazide-1-sulfonyl)phenyl]]-1-propylxanthine (PSB 0788); 4-(2,3,6,7-Tetrahydro-2,6-dioxo-1-propyl-1H-purin-8-yl)-benzenesulfonic acid (PSB 1115, CAS No. 409344-71-4); and 8-[4-[4-(4-Chlorophenzyl)piperazide-1-sulfonyl)phenyl]]-1-propylxanthine (PSB 603); [3-[4-(2,6-dioxo-1,3-dipropyl-2,3,6,7-tetrahydro-1H-purin-8-yl) (BG-9928, A1 antagonist); 8-Cyclopentyl-1,3-dipropylxanthine (DPCPX, A1 antagonist); (1S,3R)-1-[2-(6-amino-9-prop-2-ynylpurin-2-yl)ethynyl]-3-methylcyclohexan-1-ol (ATL-444, A1 and A2A antagonist); 5-Amino-7-(2-phenylethyl)-2-(2-furyl)-pyrazolo (4,3-e)-1,2,4-triazolo(1,5-c)pyrimidine (SCH-58261, A2A antagonist); 4-(2-(7-amino-2-(furan-2-yl)-[1,2,4]triazolo[1,5-a][1,3,5]triazin-5-ylamino)ethyl)phenol (ZM-241,385, A2A antagonist); 8-Ethoxy-9-ethyl-9H-purin-6-amine (ANR94, A2A antagonist); 3-ethyl-1-propyl-8-(1-(3-trifluoromethylbenzyl)-1H-pyrazol-4-yl)-3,7-dihydropurine-2,6-dione (CVT-6883, A2B antagonist); (2-(4-bromophenyl)-7,8-dihydro-4-propyl-1H-imidazo[2,1-i]purin-5 (4H)-one (KF-26777, A3 antagonist); or 3-Ethyl-5-benzyl-2-methyl-4-phenylethynyl-6-phenyl-1,4-(±)-dihydropyridine-3,5-dicarboxylateMRS-1191 (A3 antagonist).

In certain aspects a purinergic P2 receptor agonist is administered to a subject in need of an anti-cancer treatment. In a further aspect the purinergic P2 receptor agonist is an ATP analog. In certain aspects a purinergic P2 receptor agonist, e.g., ATP analog, and adenosine receptor antagonist are administered with in 1, 5, 10, 20, 30, or 60 minutes or hours of each other. In a further aspect the ATP analog and adenosine receptor antagonist are administered concurrently. In another aspect the purinergic P2 receptor agonist is administered before, during, or after administration of an adenosine receptor antagonist. In still another aspect the adenosine receptor antagonist is administered before, during, or after administration of a purinergic P2 receptor agonist.

In certain aspects a subject or patient has bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterine cancer. In a further aspect the cancer is a lung, breast, or prostate cancer. In particular aspects the cancer is a metastatic cancer, such as a bone metastasis. In certain aspects the cancer is identified as being at risk for or having a propensity for metastasis or there is no indication that the cancer has yet metastasized. In certain aspects identification of a cancer at risk of metastasis is based on assessment of a tumor biopsy.

In certain embodiments bisphosphonate drugs can be explicitly excluded from the claimed invention due to their potential in vivo toxicity.

As used herein, an “inhibitor” can be any chemical compound, peptide, or polypeptide that can reduce the activity or function of a protein. An inhibitor, for example, can inhibit directly or indirectly the activity of a protein. Direct inhibition can be accomplished, for example, by binding to a protein and thereby preventing the activity of the protein, or by inhibiting an enzymatic or other activity of the protein competitively, non-competitively, or uncompetitively. Indirect inhibition can be accomplished, for example, by binding to a protein's intended target, such as a receptor or binding partner, thereby blocking or reducing activity of the protein.

The term “effective amount” means an amount effective, at dosages and for periods of time necessary, to achieve the desired therapeutic or prophylactic result. An “effective amount” of an anti-cancer agent in reference to decreasing cancer cell growth or migration, means an amount capable of decreasing, to some extent, the growth of some cancer or tumor cells, or the inhibition of the ability of a cancer or tumor cell to migrate or invade non-tumor tissue, such as bone. The term includes an amount capable of invoking a growth inhibitory, cytostatic, and/or cytotoxic effect, and/or apoptosis of the cancer or tumor cells.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or (7) relief, to some extent, of one or more symptoms associated with the cancer or tumor. The therapeutically effective amount may vary according to factors such as the disease state, age, sex and weight of the individual and the ability of one or more anti-cancer agents to elicit a desired response in the individual. A “therapeutically effective amount” is also one in which any toxic or detrimental effects are outweighed by the therapeutically beneficial effects.

The phrases “treating cancer” and “treatment of cancer” mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or ameliorate or alleviate the symptoms of the disease caused by the cancer.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. ATP released by osteocytes treated with AD has inhibitory effect on migration of human breast cancer cells. (A) Depletion of ATP by apyrase from CM collected from osteocytes increases breast cancer cell migration. CM was collected from MLO-Y4 cells treated with (CM-AD) or without (CM) 20 μM AD for 48 hr and was then treated with or without apyrase (5 units/ml), an ATP hydrolyzing enzyme for 4 hr prior to being used to culture MDA-MB-231 cells in transwells. The cells migrated through the transwell filter were stained with Hema 3 Stat Pack (Fisher Scientific) (upper panel). The numbers of the cells migrated were quantified. Data were presented as mean±SEM, n=3. (B) CM collected from AD-treated MLO-Y4 cells has no effect on breast cancer cell proliferation. MDA-MB-231 breast cancer cells were incubated for 18 hr in CM collected from MLO-Y4 cells with (CM-AD) or without (CM) 20 μM AD for 48 hr. Data presented as mean±SEM, n=3.

FIGS. 2A-2B. The migration of human breast cancer cells is inhibited by the activation of purinergic P2X receptor. (A) oATP, a P2X antagonist, attenuates the decrease in migration of breast cancer cells when treated with CM collected from MLO-Y4 cells treated with 20 μM AD. MDA-MB-231 cells were incubated in CM collected from MLO-Y4 cells treated with (CM-AD) or without (CM) 20 μM AD for 48 hr and with or without 300 μM oATP and numbers of cells migrating in the transwell plates were quantified. Data presented as mean±SEM, n=3. (B) BzATP, a P2X7 agonist, decreases migration of human breast cancer cells. MDA-MB-231 cells were treated with various concentrations of BzATP (0-200 μM) for 48 hr and numbers of migrating cells by transwell assay were quantified. Data presented as mean±SEM, n=3. **, P<0.01; ***, P<0.001.

FIGS. 3A-3E. Antagonist of adenosine receptor and non-hydrolyzable ATP inhibit the migration of human breast cancer cells. (A) Addition of ATP increases the migration of breast cancer cells and this increase is attenuated by adenosine receptor antagonist, MRS1754. MDA-MB-231 cells were incubated in CM collected from MLO-Y4 cells treated with (CM-AD) or without (CM) 20 μM AD for 48 hr in the absence or presence of 200 μM ATP and/or 500 nM MRS1754, a potent P1 adenosine receptor antagonist. Numbers of the migrating cells by transwell assay were quantified. Data presented as mean±SEM, n=3. *, P<0.05; **, P<0.01; ***, P<0.001. (B) Lower dosage of ATP decreases, but higher dosage increases the migration of human breast cancer cells. MDA-MB-231 cells were incubated with various concentrations of ATP (0-400 μM) for 48 hr and the numbers of migrating cells by transwell assay were quantified. Data presented as mean±SEM, n=3. *, P<0.05; **, P<0.01. (C) ARL 67156 attenuates the increase in breast cancer migration from higher concentrations of ATP. MDA-MB-231 breast cancer cells were incubated with 50 μM or 200 μM ATP with or without the addition of 200 μM ARL67156. Data presented as mean±SEM, (n=3); *, P<0.05; **, P<0.01; ***, P<0.001. (D) ATPγS decreases the migration of human breast cancer cells. MDA-MB-231 breast cancer cells were incubated in CM collected from MLO-Y4 cells treated with (CM-AD) or without (CM) 20 μM AD for 48 hr and with or without 100 μM of the non-hydrolyzable ATP analogue, ATPγS. The numbers of cells by transwell assay were quantified. Data presented as mean±SEM, n=3. **, P<0.01; ***, P<0.001. (E) ATPγS decreases the migration of human breast cancer cells in a dose-dependent manner. MDA-MB-231 cells were incubated with various concentrations of ATPγS (0-400 μM) for 48 hr and the numbers of migrating cells migrated by transwell assay were quantified. Data presented as mean±SEM, n=3. **, P<0.01; ***, P<0.001.

FIGS. 4A-4B. Adenosine increases the migration of human breast cancer cells and this increase is attenuated by an adenosine receptor antagonist. (A) Adenosine and a P1 adenosine receptor antagonist increase the migration of MDA-MB-231. MDA-MB-231 breast cancer cells were incubated in CM collected from MLO-Y4 cells treated with (CM-AD) or without (CM) 20 μM AD for 48 hr in the absence or presence of 200 μM adenosine and/or 500 nM of MRS 1754. The numbers of migrating cells by transwell assay were quantified. Data is presented as mean±SEM, n=3. **, P<0.01; ***, P<0.001. (B) The increased migration of breast cancer cells by apyrase is attenuated by MRS 1754. MDA-MB-231 breast cancer cells were incubated in CM collected from MLO-Y4 cells treated with (CM-AD) or without (CM) 20 μM AD for 48 hr and then treated with or without apyrase (5 units/ml) and or/or 500 nM MRS 1754. Data presented as mean±SEM, n=3. The numbers of migrating cells by transwell assay were quantified. *, P<0.05; **, P<0.01; ***, P<0.001.

FIG. 5. The anchorage-independent growth of human breast cancer cells is inhibited by ATPγS, but stimulated by adenosine. MDA-MB-231 breast cancer cells were plated on soft agar and were treated with 100 μM ATPγS, 200 μM adenosine, or without for about 2 weeks. Cells growing on soft agar plates were imaged (upper panel) and quantified (lower panel). Data presented as mean±SEM, n=3. *, P<0.05; **, P<0.01.

FIGS. 6A-6C. The reduction of mouse mammary cancer cells by ATP and ATPγS. (A) ATP reduced the migration of murine mammary cancer cells in a dose-dependent manner. Py8119 mouse mammary cancer cells were incubated in CM collected from MLO-Y4 cells treated with (CM-AD) or without (CM) 20 μM AD for 48 hr and ATP in concentrations ranging from 0-400 μM. Data presented as mean±SEM, n=3. (B) ATPγS decreased the migration of murine mammary cancer cells. Py8119 mouse mammary cancer cells were incubated in CM collected from MLO-Y4 cells treated with (CM-AD) or without (CM) 20 μM AD for 48 hr in the absence or present of 100 μM ATPγS or 200 μM ATP. Data presented as mean±SEM, n=3. (C) Adenosine has no effect on murine mammary cancer cell migration. Py8119 cells were incubated with various concentrations of adenosine (0-40 μM) for 48 hr and numbers of migrating cells by transwell assay were quantified. Data presented as mean±SEM, n=3.

FIGS. 7A-7B. Systemic administration of ATPγS reduces the growth of MDA-MB-231 mammary cells in vivo. MDA-MB-231 cells were injected into the mammary fat pads of nude female mice at 1×10⁶ cells per mouse. The mice were treated three times a week IP with 500 μl of saline or saline containing 400 μmol of ATPγS or adenosine. (A) Tumor volumes were calculated with the equation V=(L×W²)×0.5 (mm³), where L is length and W is width of a tumor (n=14 measurements per group). Data presented as mean±SEM; saline vs ATPγS at 17 days, *, P<0.05; saline vs ATPγS at 21 days, ***, P<0.001; saline vs adenosine at 17 days, *, P<0.05; saline vs adenosine at 21 days, **, P<0.01. (B) Left: Photomicrographs of orthotopic tumors excised from mice. Right: Tumor volume of the orthotopic tumor tissues from saline or ATPγS treated mice. Data presented as mean±SEM (n=14 per group); saline vs ATPγS average tumor weight, **, P<0.01; saline vs adenosine average tumor weight, *, P<0.05; adenosine vs ATPγS average tumor weight, ***, P<0.001.

FIGS. 8A-8B. Systemic administration of ATPγS reduces the growth of Py8119 mammary carcinoma cells in bone. Py8119/Luc-GFP cells were injected into the right tibias of WT female mice at 1×10⁵ cells per mouse. The mice were treated three times a week IP with 500 μl of saline or saline containing 400 μmol of ATPγS. (A) Whole body imaging analysis of mice (n=5 per group). Both ventral and dorsal views are shown. (B) Total photon flux was taken once a week after tumor cell injection. Luciferase signals were quantified by using Living Image 3.2. Data presented as mean±SEM (n=5 per group); 4 weeks ventral view, *, P<0.05; 4 weeks dorsal view, **, P<0.01.

FIG. 9. Illustrates results from a transwell migration assay and the effects of A2A receptor antagonist on MDA-MB-231 breast cancer cell migration.

### DESCRIPTION

Skeletal metastases in patients have been characterized as osteolytic, osteoblastic or both, and in all cases, there is a disruption of the normal bone remodeling process (Roodman, (2004) N. Engl. J. Med. 350, 1655-1664). In addition, there is a close relationship between bone destruction and tumor growth. There are three major cell types in bone tissues: osteocytes, osteoblasts, and osteoclasts. Osteocytes comprise over 95% of total bone cells and play an essential role in orchestrating the bone remodeling process by coordinating activities from the osteoclasts and osteoblasts (Bonewald, (2007) Ann. N. Y. Acad. Sci. 1116, 281-290; Matsuo, (2009) Curr. Opin. Nephrol. Hypertens. 18, 292-297). The roles of osteoblasts and osteoclasts in bone metastasis have been linked to the release of growth factors from the bone matrix, which stimulates tumor growth (Roodman, (2004) N. Engl. J. Med. 350, 1655-1664). However, the role of osteocytes, the most abundant cell type in bone tissue, in bone metastases remains unexplored.

The growth and migration of tumor cells are largely influenced by its microenvironment and bone is one of the most preferred sites for cancer metastasis. Bone cells are reported to release various cytokines and growth factors that influence the behavior of cancer cells (Roodman, (2004) N. Engl. J. Med. 350, 1655-1664). Osteocytes are known to release several factors, including prostaglandin, nitric oxide, and ATP by mechanical stimulation (Batra et al., (2012) Biochim. Biophys. Acta. 1818, 1909-1918). Thus far, bisphosphonates are the primary drugs used for the treatment of cancer metastasis to the bone.

The inventors describe herein that ATP released by osteocytes associated with the activation of purinergic receptor(s) is responsible for the inhibitory effect of bisphosphonates on breast cancer cell migration. In contrast, adenosine and adenosine receptor(s) have stimulatory effect on breast cancer cell migration.

Although osteocytes comprise over 95% of total cells in the bone, their involvement in cancer bone metastasis is not fully understood. Moreover, the mechanism underlying the inhibitory effect of bisphosphonates on bone metastasis is also largely unexplored. The inventors observed that conditioned medium (CM) collected from alendronate (AD)-treated osteocytes decreased numbers of breast cancer cells migrating to the other side of the transwell filter. This decrease is caused by the reduction of cell migration, but not total number of cells as WST-1 assay failed to detect any alteration in cell proliferation. The inhibitory effect is likely to be mediated by ATP since depletion of ATP by apyrase or application of antagonist of P2X receptors completely attenuated such effect. The direct treatment with ATP inhibits migration. However, the inventors observed that addition of ATP enhances, instead of reducing, the migration MDA-MB-231 breast cancer cells. Extracellular ATP is unstable and can be hydrolyzed by ectonucleotidase released from the cell (Deli and Csernoch, (2008) Pathol. Oncol. Res. 14, 219-231). The inventors contemplate that hydrolysable products of ATP, such as adenosine exert an opposite effect from ATP on cancer cell migration. Indeed, treatment of non-hydrolysable ATP, ATPγS, and an adenosine receptor antagonist MRS1754 significantly attenuated this adverse effect.

Extracellular nucleotides and nucleosides have been shown to participate in signal transduction through purinergic receptors and affect a variety of cellular functions and processes such as inflammation, development and regeneration, and cancer (Burnstock, (2008) J. Physiol. 586, 3307-3312). In accordance with our findings, published studies have indicated biphasic effects of ATP on cancer cells. Many studies indicate the action of ATP on P2 purinergic receptors to cause an anticancer effect (White and Burnstock, (2006) Trends Pharmacol. Sci. 27, 211-217). On the other hand, other studies have shown that activation of P2 receptors in some breast cancer cell lines could cause an increase in cell migration (Jelassi et al., (2011) Oncogene 30, 2108-2122). This discrepancy could possibly be due to varying expression levels of P2 ATP receptors reported among different breast cancer cells types. Additionally, there is increased expression of certain P2X receptors in breast tissue undergoing malignant change compared to normal breast tissue (White and Burnstock, (2006) Trends Pharmacol. Sci. 27, 211-217). Consistent with the currently described observation of human breast and mouse mammary cancer cells, a similar stimulatory effect of adenosine on cancer cell chemotaxis has been observed previously for A2058 melanoma cells and this response was inhibited by adenosine receptor antagonists (Woodhouse et al., (1998) Biochem. Biophys. Res. Commun. 246, 888-894). Bladder and prostate carcinomas seem to be inhibited by the activation of the P1 adenosine receptors, and anti-proliferative, pro-apoptotic, and pro-necrotic effects have been reported in several other different cell types (Rapaport et al., (1983) Cancer Res. 43, 4402-4406; Shabbir and Burnstock, (2009) Int. J. Urol. 16, 143-150). It has also been reported that human primary breast tumor tissues express higher levels of P1 adenosine receptors than in matched normal breast tissues (Gessi et al., (2011) Biochim. Biophys. Acta. 1808, 1400-1412).

The inventors sought to confirm the results described herein by using a different mammary carcinoma cell line from mouse, Py8119. Like human breast cancer cells, the inventors found that the treatment with adenosine can similarly promote cell migration and this enhancement is inhibited by MRS1754. This antagonist blocks adenosine A2B receptor signaling, suggesting the importance of this receptor in breast cancer cell migration. Based on the effect of the antagonist in two types of breast cancer cells, A2B receptor could be a major receptor in mediating the effect of adenosine in promoting breast cancer migration. Together, the studies point to the differentiation roles of adenosine nucleotides and purinergic receptors in tumor invasion and metastasis, and imply the use these purinergic receptors as targets in cancer metastasis therapeutics.

### I. PURINERGIC RECEPTORS AND ANTAGONIST THEREOF

Purinergic receptors, also known as purinoceptors, are a family of plasma membrane polypeptides involved in several cellular functions such as vascular reactivity, apoptosis, and cytokine secretion. These functions have not been well characterized and the effect of the extracellular microenvironment on their function is also poorly understood. The term purinergic receptor was originally introduced to illustrate specific classes of membrane receptors that mediate relaxation of gut smooth muscle as a response to the release of ATP (P2 receptors) or adenosine (P1 receptors). P2 receptors have further been divided into five subclasses: P2X, P2Y, P2Z, P2U, and P2T. To distinguish them further, the subclasses have been divided into families of metabotropic (P2Y, P2U, and P2T) and ionotropic receptors (P2X and P2Z).

1. ATP (P2 Purinergic) Receptor Ligands

P2 purinergic receptors are positively modulated by agonist such as ATP analogs (e.g., non-hydrolysable ATP analogs). ATP has long been known to play a central role in the energetics of cells both in transduction mechanisms and in metabolic pathways, and is involved in regulation of enzyme, channel, and receptor activities. Numerous ATP analogs have been synthesized to probe the role of ATP in biosystems. Modifications can be introduced in the phosphate chain of ATP that significantly diminish the ability of enzymes and receptors to hydrolyze the compound. Such non-hydrolysable ATP analogs competitively inhibit ATP-dependent enzyme systems, such as purinergic receptors.

In certain aspects, ATP analogs include, but are not limited to adenosine 5′-[α-thio]triphosphate (ATPαS); alpha,beta-methylene-adenosine-5′-diphosphate (ApCpp); beta,gamma-methylene-ATP (AppCp); adenosine 5′[γ-thio]triphosphate (ATPγS); adenylyl imidodiphosphate (AMP-PNP); N⁶-diethyl-beta,gamma-dibromomethylene-ATP; 2-methylthio-ATP (APM); alpha,beta-methylene-ATP; beta,gamma-methylene-ATP; di-adenosine pentaphosphate (Ap5A); 1,N⁶-ethenoadenosine triphosphate; adenosine 1-oxide triphosphate; 2′,3′-O-(benzoyl-4-benzoyl)-ATP (B-ZATP); and 2′,3′-O-(2,4,6-trinitrophenyl)-ATP (TNP-ATP), the various structures of which can be found in the PubChem database on the world wide web at ncbi.nlm.nih.gov/pccompound (non-hydrolysable analogs can be purchased, for example, from Jena Biosciences, Jena, Germany; Sigma-Aldrich, St. Louis, Mo., USA).

2. Adenosine (P1 Purinergic) Receptor Antagonist

In humans, there are four types of adenosine receptors. Each is encoded by a separate gene and has different functions, although with some overlap. For instance, both A1 receptors and A2A play roles in the heart, regulating myocardial oxygen consumption and coronary blood flow, while the A2A receptor also has broader anti-inflammatory effects throughout the body. These two receptors also have important roles in the brain, regulating the release of other neurotransmitters such as dopamine and glutamate, while the A2B and A3 receptors are located mainly peripherally and are involved in processes such as inflammation and immune responses.

Some compounds acting on adenosine receptors are nonselective, with the endogenous agonist adenosine being used in hospitals as treatment for severe tachycardia (rapid heart beat), and acting directly to slow the heart through action on all four adenosine receptors in heart tissue, as well as producing a sedative effect through action on A1 and A2A receptors in the brain. Xanthine derivatives such as caffeine and theophylline act as non-selective antagonists at A1 and A2A receptors in both heart and brain and so have the opposite effect to adenosine, producing a stimulant effect and rapid heart rate.

Other adenosine receptor agonists and antagonists are much more potent and subtype-selective, and have allowed extensive research into the effects of blocking or stimulating the individual adenosine receptor subtypes, which is now resulting in a new generation of more selective drugs with many potential medical uses. Some of these compounds are still derived from adenosine or from the xanthine family, but researchers in this area have also discovered many selective adenosine receptor ligands that are entirely structurally distinct, giving a wide range of possible directions for future research.

Certain aspects utilize antagonist of the adenosine A2B receptor. Adenosine receptor antagonist include, but are not limited to N-(4-Cyanophenyl)-2-[4-(2,3,6,7-tetrahydro-2,6-dioxo-1,3-dipropyl-1H-purin-8-yl)phenoxy]-acetamide (MRS 1754, CAS no. 264622-58-4); N-(4-Acetylphenyl)-2-[4-(2,3,6,7-tetrahydro-2,6-dioxo-1,3-dipropyl-1H-purin-8-yl)phenoxy]acetamide (MRS 1706, CAS No. 264622-53-9); 8-[4-[4-(4-Chlorobenzyl)piperazide-1-sulfonyl)phenyl]]-1-propylxanthine (PSB 0788); 4-(2,3,6,7-Tetrahydro-2,6-dioxo-1-propyl-1H-purin-8-yl)-benzenesulfonic acid (PSB 1115, CAS No. 409344-71-4); and 8-[4-[4-(4-Chlorophenzyl)piperazide-1-sulfonyl)phenyl]]-1-propylxanthine (PSB 603); [3-[4-(2,6-dioxo-1,3-dipropyl-2,3,6,7-tetrahydro-1H-purin-8-yl) (BG-9928, A1 antagonist); 8-Cyclopentyl-1,3-dipropylxanthine (DPCPX, A1 antagonist); (1S,3R)-1-[2-(6-amino-9-prop-2-ynylpurin-2-yl)ethynyl]-3-methylcyclohexan-1-ol (ATL-444, A1 and A2A antagonist); 5-Amino-7-(2-phenylethyl)-2-(2-furyl)-pyrazolo(4,3-e)-1,2,4-triazolo(1,5-c)pyrimidine (SCH-58261, A2A antagonist); 4-(2-(7-amino-2-(furan-2-yl)-[1,2,4]triazolo[1,5-a][1,3,5]triazin-5-ylamino)ethyl)phenol (ZM-241,385, A2A antagonist); 8-Ethoxy-9-ethyl-9H-purin-6-amine (ANR94, A2A antagonist); 3-ethyl-1-propyl-8-(1-(3-trifluoromethylbenzyl)-1H-pyrazol-4-yl)-3,7-dihydropurine-2,6-dione (CVT-6883, A2B antagonist); (2-(4-bromophenyl)-7,8-dihydro-4-propyl-1H-imidazo[2,1-i]purin-5 (4H)-one (KF-26777, A3 antagonist); and 3-Ethyl-5-benzyl-2-methyl-4-phenylethynyl-6-phenyl-1,4-(±)-dihydropyridine-3,5-dicarboxylateMRS-1191 (A3 antagonist).

B. Targeting

Targeting moieties can be used to allow the therapeutic agent(s) to bind to proteins or other targets associated with a cancer and increase the concentration of the agent(s) at a site to be treated. In one embodiment, the targeting moiety can be a molecule, peptide, or a protein (e.g., antibody) suitable to target certain receptors or cells. The particular targeting moiety useful with this invention can be dependent on the nature of the target and the specific requirements of the binding. Therapeutic agent(s) can be directly or indirectly coupled to a cancer targeting moiety. In certain aspects the therapeutic agent(s) are comprised in a liposome having a cancer targeting moiety associated with the liposome. In certain aspects the targeting moiety is a peptide, antibody, or antibody fragment that selectively associates with a cancer cell or tumor. In a further aspect a therapeutic agent can be directly coupled to a targeting moiety. In certain aspects the therapeutic agent can be reversibly coupled so that the therapeutic agent and the targeting moiety disassociate at the site to be treated.

The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. Binding also defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

In one embodiment, the targeting moiety may be selected for the ability to interact with a receptor expressed on specific types of cells or tissue and to induce endocytosis. For example, such cells may be targeted to cell biomarkers or cancer biomarkers which are specific receptors expressed on the surface at specific densities. Further, these receptors or biomarkers are shown in the literature and are consistently being discovered and reported thereon. One of ordinary skill in the art may select targeting peptides without undue experimentation by reviewing the literature to finding peptides that can bind and induce endocytosis in specific types of cells.

Suitable targeting moieties include, but are not limited to peptides or proteins that are able to bind to specific types of cells or tumors. Such targeting moieties may be ligands that can target receptors on specific cancers. For example, the targeting moiety may be somatostatin, which can target somatostatin receptors subtypes sstl-5 found in human neuroendocrine tumors and other lymphomas. Other suitable targeting moieties may be small molecules such as folic acid or carbohydrates, phosphorylated peptides and glycoproteins or peptides. Suitable targeting moieties include, but are not limited to cell surface binding peptides (e.g., RGD peptide and NGR peptide), molecular ligands (e.g., folate), polypeptide ligands (e.g., transferrin and GM-CSF), sugars and carbohydrates (e.g., galactosoamine), and antibodies (e.g., anti-VEGFR, anti-ERBB2, anti-tenascin, anti-CEA, anti-MUC1, or anti-TAG72). In certain embodiments these targeting moieties are coupled to a liposome. In other embodiments the targeting moieties are coupled to the therapeutic agents.

Tumor associated antigens that can be used in targeting include, but are not limited to gp100, Melan-A/MART, MAGE-A, MAGE (melanoma antigen E), MAGE-3, MAGE-4, MAGEA3, tyrosinase, TRP2, NY-ESO-1, CEA (carcinoembryonic antigen), PSA, p53, Mammaglobin-A, Survivin, Mucl (mucin1)/DF3, metallopanstimulin-1 (MPS-1), Cytochrome P450 isoform 1B1, 90K/Mac-2 binding protein, Ep-CAM (MK-1), HSP-70, hTERT (TRT), LEA, LAGE-1/CAMEL, TAGE-1, GAGE, 5T4, gp70, SCP-1, c-myc, cyclin B1, MDM2, p62, Koc, IMP1, RCAS1, TA90, OA1, CT-7, HOM-MEL-40/SSX-2, SSX-1, SSX-4, HOM-TES-14/SCP-1, HOM-TES-85, HDAC5, MBD2, TRIP4, NY-CO-45, KNSL6, HIP1R, Seb4D, KIAA1416, IMP1, 90K/Mac-2 binding protein, MDM2, NY/ESO, and LMNA.

### II. TREATMENT OF CANCER

The inventors have shown that modulating ATP and/or adenosine related pathways can be used to inhibit proliferation and/or migration of cancer cells. In certain aspects the cancer is a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterine cancer. In a further aspect the cancer is breast cancer. In still a further aspect the cancer is prostate cancer. In particular embodiments the cancer is metastatic cancer, e.g., cancer that has or is at risk of metastasizing or migrating to the bone.

In certain embodiments, the invention also provides compositions comprising one or more anti-cancer agents in a pharmaceutically acceptable formulation. Thus, the use of one or more anti-cancer agents that are provided herein in the preparation of a medicament is also included. Such compositions can be used in the treatment of a variety of cancers. In certain embodiments the treatment is for a metastatic cancer, e.g., lung, breast, or prostate cancer.

The anti-cancer agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-cancer agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor or a metastasis in question is also contemplated by the present invention. When administering the compositions by injection, the administration may be by continuous infusion or by single or multiple boluses. For parenteral administration, the agents may be administered in a pyrogen-free, parenterally acceptable aqueous solution comprising the desired anti-cancer agents in a pharmaceutically acceptable vehicle. A particularly suitable vehicle for parenteral injection is sterile distilled water in which one or more anti-cancer agents are formulated as a sterile, isotonic solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight. In certain aspects, non-hydrolysable ATP analogs can be administered by infusion to patients in daily dosages at rates ranging from 20, 25, 30, 35, 40 to 30, 35, 40, 45, 50 μg/kg/min (including all values and ranges there between) for up to 8 hours, including 1, 2, 3, 4, 5, 6, 7, or 8 hours. Non-hydrolysable ATP analogs can be administered orally at about 1, 10, 20, 30, 40, 50, 60 to 50, 60, 70, 80 90, 100 μg/kg or mg/kg of body weight per day. In certain aspects the non-hydrolysable ATP analog can be administered at about 0.01 to 10 mg/kg of body weight per day.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy or radiotherapy, which may be administered more than one time. Chemotherapy includes, but is not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof. Radiation therapy includes, but is not limited to, X-ray irradiation, UV-irradiation, γ-irradiation, electron-beam radiation, or microwaves. Moreover, a cell or a patient may be administered a microtubule stabilizing agent, including, but not limited to, taxane, as part of methods of the invention. It is specifically contemplated that any of the compounds or derivatives or analogs, can be used with these combination therapies.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Results

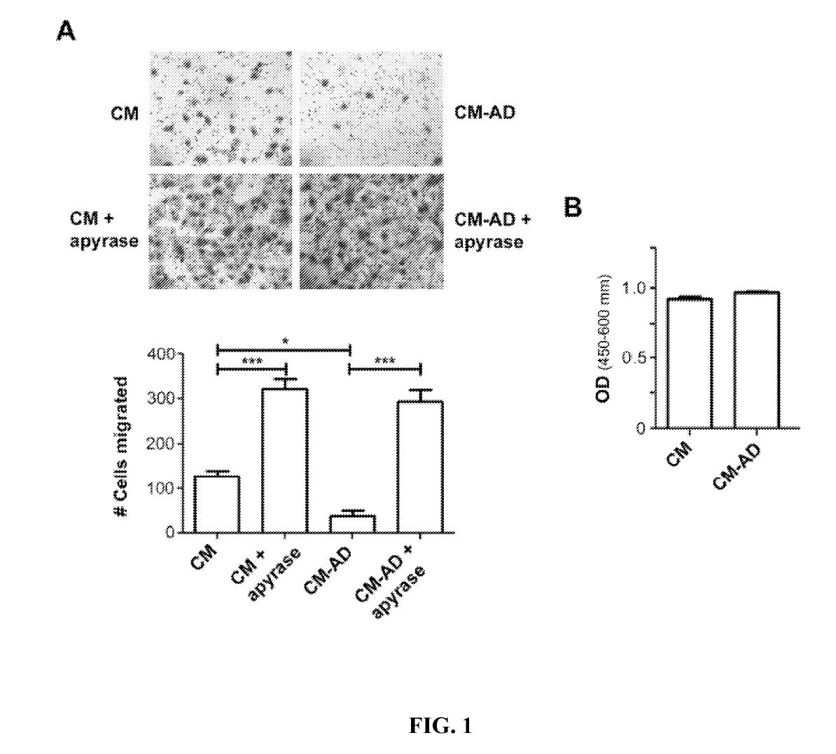

ATP released by AD-treated osteocytes inhibits the migration of human breast cancer cells. To determine the underlying mechanism of the bisphosphonates in suppressing cancer metastasis to the bone, the inventors treated osteocytic MLO-Y4 cells with AD and collected CM. The result from the transwell cell migration assay showed that CM collected from the MLO-Y4 osteocytes treated with AD significantly decreased the migration of MDA-MB-231 cells (from 127±12 cells to 38±12 cells) (FIG. 1A). To eliminate the possibility of any effects from proliferation, the WST-1 cell proliferation assay was performed by incubating the MDA-MB-231 breast cancer cells in the identical CM and time duration as used in the transwell migration assay. The proliferation of the MDA-MB-231 cells incubated in CM from MLO-Y4 cells treated with 20 μM AD (CM-AD) was similar to that of the MDA-MB-231 cells incubated in untreated CM (CM) (FIG. 1B). To determine whether ATP released from osteocytes would have an effect on MDA-MB-231 cell migration, the inventors depleted ATP from the CM collected from MLO-Y4 cells using apyrase, an ATP hydrolyzing enzyme. The addition of apyrase increased MDA-MB-231 cell migration by 2.5 fold in untreated CM and 7.7 fold in CM-AD (FIG. 1A). These results suggest that ATP released from osteocytes upon AD treatment can inhibit the migration of human breast cancer cells.

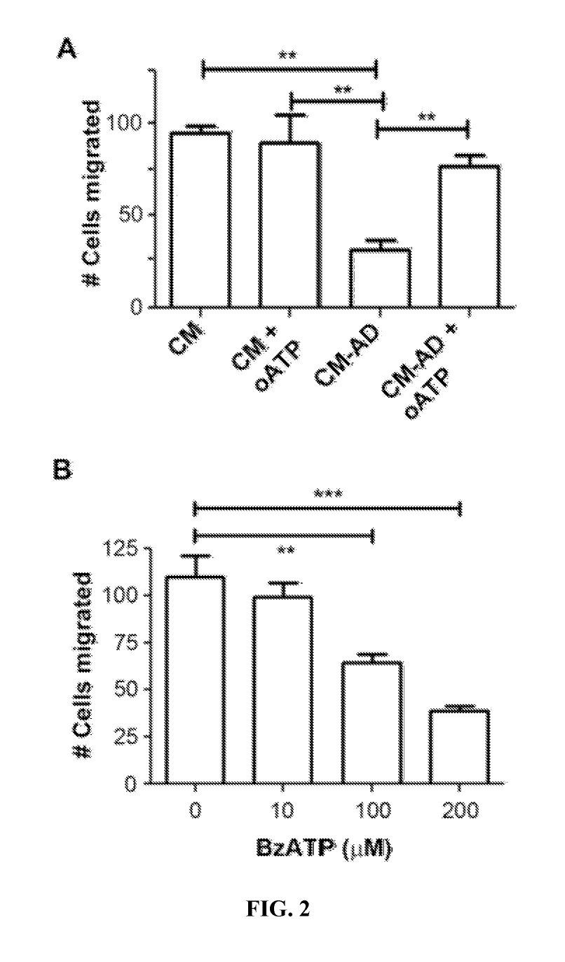

To test the effect of purinergic signaling activated by ATP on breast cancer cell migration, the inventors treated the CM with oxidized ATP (oATP), a potent inhibitor of P2X purinergic receptors. The addition of oATP significantly attenuated the inhibitory effect of CM-AD on MDA-MB-231 cell migration (FIG. 2A). Consistently, the addition of BzATP, a nonhydrolyzable P2X7 receptor agonist, caused a significant, dose-dependent decrease in breast cancer migration (0 μM=110±11.6 cells, 10 μM=99±7.8 cells, 100 μM=64±4.4 cells, 200 μM=39±2.6) (FIG. 2B). The result from the WST-1 assay showed that the treatment with BzATP at concentrations 1-200 μM had minimal effects on cell proliferation, and a significant reduction was only observed at 400 μM. These data support an inhibitory role of P2X receptor activation in the migration of human breast cancer cell.

ATP Inhibits, but Adenosine Promotes the Migration of Breast Cancer Cells.

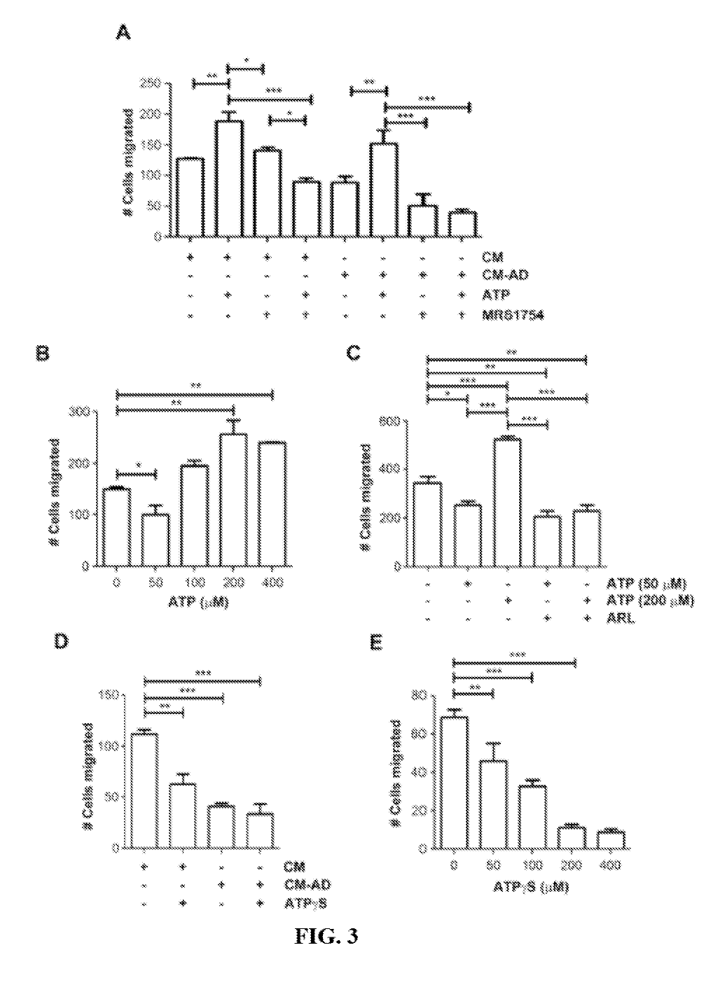

To determine the direct involvement of ATP, the inventors applied ATP into the CM. Surprisingly, the treatment of ATP did not decrease, but increased the migration of MDA-MD-231 cells in both CM collected from AD and non-AD-treated MLO-Y4 cells (153±21.1 vs. 88±10.7 and 188±33.5 vs. 127±2, respectively) (FIG. 3A). To further test the effect of ATP, the inventors treated MDA-MB-231 cells with ATP at varying concentrations (FIG. 3B). The inventors found that the inhibitory effect of ATP was only observed at lower concentration (0 μM=150±4.8 cells vs 50 μM=100±17.7 cells), but higher concentration instead promoted cancer cell migration (200 μM=257±26 cells, 400 μM=240±0.9 cells). The effect of ATP on cell migration was not caused by alterations of cell proliferation. This is possibly due to higher levels of adenosine formed as a product of the increased break down of ATP at higher concentrations, since extracellular ATP is known to be readily hydrolyzed to adenosine by a group of enzymes known as ectonucleotidases (Deli and Csernoch, Pathol Oncol Res, 2008; 14: 219-31). To test for the possible effects of adenosine as a result of ATP hydrolysis, a potent adenosine receptor antagonist, MRS1754 was used. The addition of MRS1754 attenuated the stimulatory effect of ATP on the migration (FIG. 3A). Moreover, MRS1754 further augmented the inhibitory effect of CM-AD on cell migration, suggesting these adverse effects were mediated by adenosine. As further confirmation, the inventors applied an ecto-ATPase inhibitor, ARL67156, which prevents the breakdown of ATP. The addition of ARL67156 attenuated the stimulatory effect of higher dosage of ATP on the migration of the breast cancer cells (FIG. 3C). However, the effect of ARL67156 on cell migration was not caused by changes in cell proliferation.

To demonstrate the effect of ATP in the absence of its break down, the inventors used a nonhydrolyzable ATP analogue, ATPγS. The application of this reagent to control CM significantly reduced the migration of MDA-MB-231 cells (112±2.4 cells to 63±9.6 cells) (FIG. 3D). The significant reduction of cancer cell migration by ATPγS was further demonstrated in a dose-dependent manner (0 μM=69±3.8 cells, 50 μM=46±9.3 cells, 100 μM=33±3.3 cells, 200 μM=11±1.7, 400 μM=9±1.5 cells) (FIG. 3E). This data confirms the inhibitory role of ATP on breast cancer cell migration and implies the opposite role of adenosine.

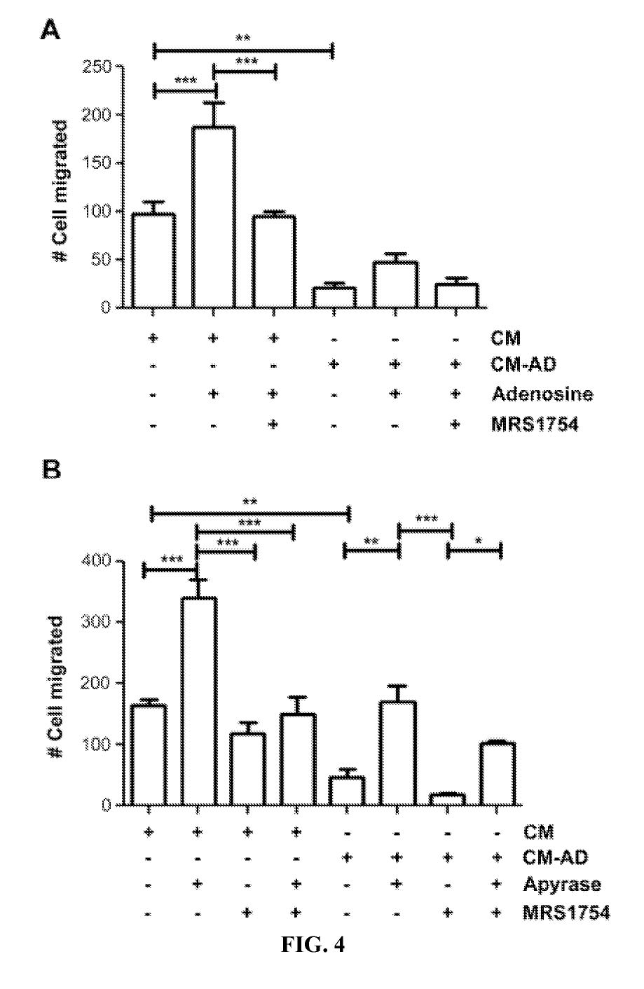

The inventors further tested the effect of adenosine on MDA-MB-231 cell migration. CM collected from MLO-Y4 cells were treated with or without adenosine. The adenosine receptor antagonist MRS1754 was also added to verify the specific effect from adenosine. The treatment of adenosine increased MDA-MB-231 cell migration, whereas this increase was completely attenuated with the addition of MRS1754 (FIG. 4A). The enhanced cell migration by adenosine was not a result of increased cell proliferation since the treatment of adenosine at various concentrations had minimal effects on cell proliferation. To further determine if a similar effect was also observed with the hydrolysis of ATP, the inventors added apyrase to the CM. Consistently, the increase of the migration as a result of apyrase treatment was significantly attenuated by MRS1754 (FIG. 4B). Based on these data, the inventors concluded that adenosine has a stimulatory role on breast cancer cell migration and this effect is mediated through adenosine receptor signaling. These results further suggest the divergent roles of ATP and adenosine on breast cancer cell migration; the inhibitory role by ATP and the stimulatory role by adenosine.

ATPγS Inhibits, but Adenosine Promotes Anchorage-Independent Growth of Human Breast Cancer Cells.

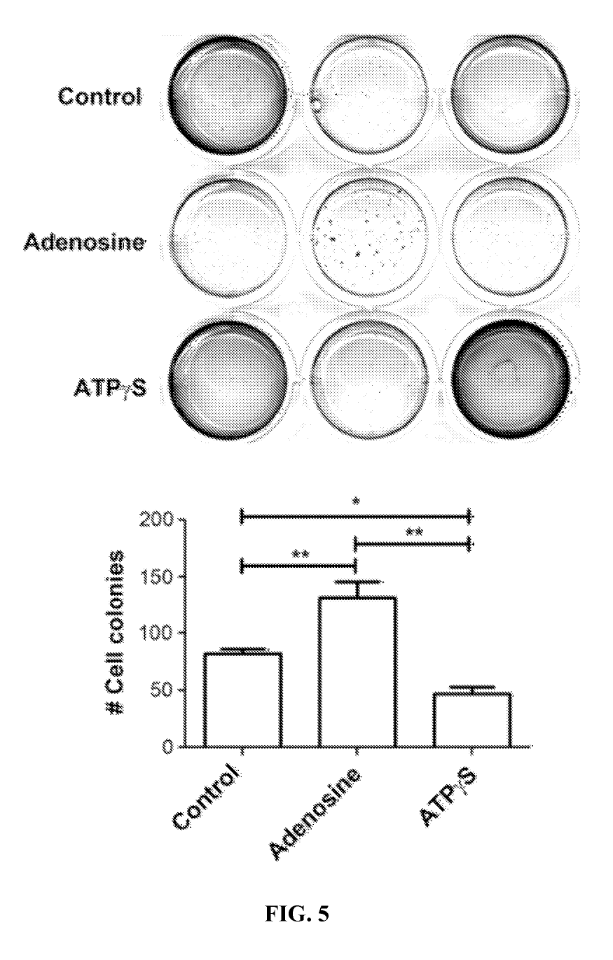

To determine if ATP and adenosine have similar effects on the anchorage-independent growth of human cancer cells, the inventors cultured MDA-MB-231 breast cancer cells in soft agar (FIG. 5). Similar to their effects on the cell migration, ATPγS significantly inhibited colony formation of MDA-MB-231 cells (82±4.5 colonies to 47±6.2 colonies), while adenosine had an opposite effect by significantly promoting colony formation (132±13.7 colonies). These results suggest that ATP and adenosine not only affect cell migration, but also have a major impact on human cancer cell growth.

ATP and ATPγS Inhibited the Migration of Mouse Mammary Carcinoma Cells.

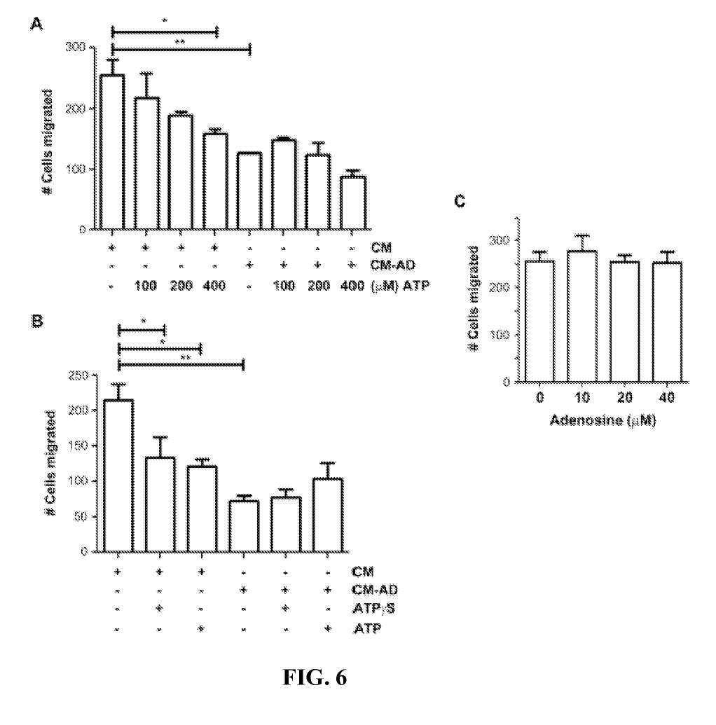

The inventors tested adenosine nucleotides on Py8119, a mouse mammary carcinoma cell line, since this cell is capable of metastasizing to other tissues in non-immunodeficient wild-type mice and has been used as an in vivo metastatic model (Deli and Csernoch, Pathol Oncol Res, 2008; 14: 219-31). ATP at varying concentrations was added to the CM collected from MLO-Y4 cells treated with (CM-AD) or without (CM) 20 μM AD. The transwell cell migration assay was conducted with Py8119 cells incubated in these CM. With the increase of dosage, the migration of the Py8119 cancer cells decreased, with the most significant effect at 400 μM (254±25.9 cells to 159±7.8 cells with CM and 127±0.3 cells to 88±10.3 cells for CM-AD) (FIG. 6A). The migration of Py8119 cells was also decreased with the treatment of ATPγS (FIG. 6B). These results suggest that similar to MDA-MB-231 breast cancer cells, ATP has an inhibitory role on Py8119 mouse mammary carcinoma cells and further implies a broad role of ATP on breast cancer bone metastasis. The inventors then tested whether adenosine has a similar stimulatory effect on the Py8119 cells as it had on the MDA-MB-231 cells. The transwell migration assay was conducted with Py8119 cells incubated in media containing various concentrations of adenosine (FIG. 6C). The inventors found that the migration of Py8119 cells was not changed, regardless of the concentration of adenosine added. This indicates that unlike the human breast cancer cell line MDA-MB-231, the migration of the mouse mammary carcinoma cell line Py8119 is not sensitive to adenosine.

ATPγS Inhibited the Tumor Growth of Human Mammary Carcinoma Cells in Nude Mouse Xenografts.

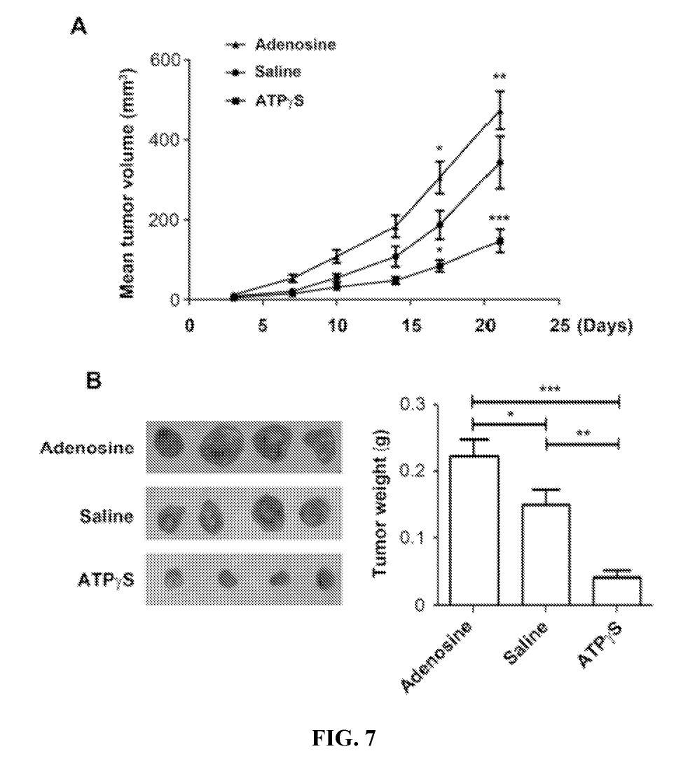

The in vitro data demonstrated the inhibitory effect of ATP on breast cancer cell growth and migration. To test if ATP has a similar, inhibitory effect on tumor growth in vivo, the inventors used an orthotopic mouse model. MDA-MB-231 cells were orthotopically implanted into the mammary fat pads of athymic female nude mice. After the mice were randomly assigned into 3 different treatment groups, the mice were treated with or without ATPγS or adenosine. The ATPγS and adenosine were administered through IP injections at 400 μmol per mouse three times a week. The control mice were injected IP with saline. Dosages were determined by a previous study showing no toxicity from IP injections of up to 50 mM of adenine nucleotides into mice for 10 days. Tumor sizes were measured once every three to four days throughout the treatment period. At the end of the study, the tumors were excised and weighed. The inventors found that the mice treated with ATPγS exhibited significantly reduced tumor growth rate in comparison to the control group, while the adenosine treated mice had an increase in tumor growth rate (FIG. 7A). The reduced mean tumor volume of the treatment group was statistically significant after 17 days of ATPγS treatment. In post mortem analysis, the tumors excised from the mammary fat pads showed significantly (over 4 fold) decreased sizes in the ATPγS-treated group as compared to the control group. Additionally, the adenosine-treated group had 50% increased tumor sizes compared to the control group tumors (FIG. 7B). These results reveal that systemic administration of ATPγS had an inhibitory effect on the growth of human breast cancer cells in vivo.

ATPγS Inhibited the Tumor Growth and Metastasis of Mouse Mammary Carcinoma Cells In Vivo.

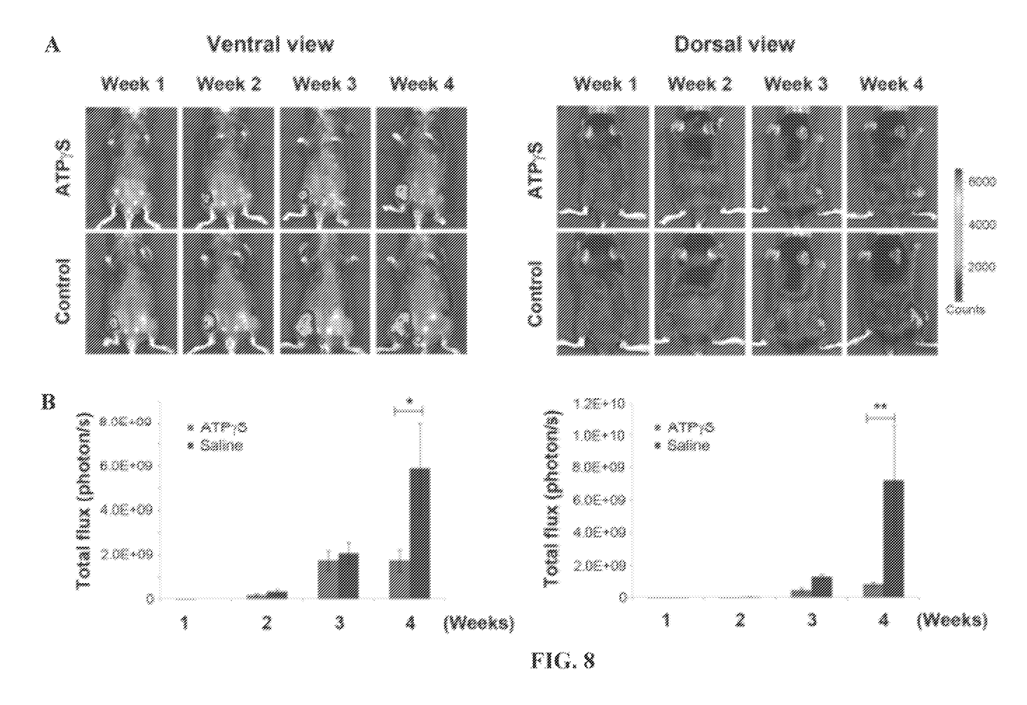

To assess how systemic treatment with ATPγS may affect the growth of breast cancer cells in the bone microenvironment of a syngeneic host, the inventors performed intratibial injections in wild-type C57b1/6 female mice using the mouse mammary carcinoma cell line Py8119. The mammary tumor cells were injected into the right tibias of the female mice, and the tumor growth was monitored with whole animal imaging once a week for 4 weeks. The mice were treated with IP injection of saline supplemented with or without 400 μmol ATPγS three times a week. Bioluminescence analysis of the animals revealed that treatment with ATPγS significantly inhibited tumor growth in the tibias (FIG. 8). Results indicate that mice injected with ATPγS had a dramatic reduction in tumor burden after 4 weeks of treatment as reflected by bioluminescence signals from the images taken in both the dorsal (right panels) and ventral (left panels) positions. Quantification data (lower panels) further confirmed the significant decrease of tumor growth in bone with the treatment of ATPγS.

Attenuation of Cell Migration by A2A Antagonist.

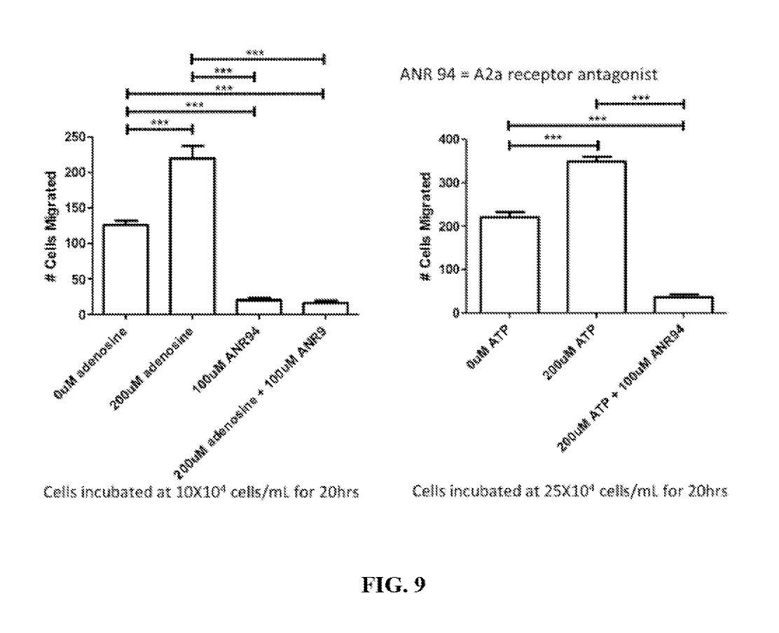

Adenosine increases the migration of human breast cancer cells and this increase is attenuated by an A2A receptor antagonist (FIG. 9). The increased migration of breast cancer cells by adenosine is attenuated by ANR94. MDA-MB-231 breast cancer cells were incubated in the presence of 200 μM adenosine and/or 100 μM of ANR94 for 20 hr. The numbers of migrating cells by transwell migration assay were quantified. The increased migration of breast cancer cells by ATP is attenuated by ANR94. MDA-MB-231 breast cancer cells were incubated in the presence of 200 μM ATP and/or 100 μM of ANR94 for 20 hr. The numbers of migrating cells by transwell migration assay were quantified.

B. Materials & Methods

Materials.

MLO-Y4 osteocytic cells derived from mouse long bones were kindly provided by Lynda Bonewald (University of Missouri at Kansas City). Alendronate (4-amino-1-hydroxybutylidene-1,1-bisphosphonic acid), ATP, ATPγS (adenosine 5′-[γ-thio]triphosphate tetralithium salt), BzATP (2′(3′)-O-(4-Benzoylbenzoyl)adenosine-5′-triphosphate tri(triethylammonium) salt), oxidized ATP (oATP), adenosine, apyrase, and MRS 1754 were purchased from Sigma. ARL67156 was purchased from R&D systems.

Cell Lines and Cell Cultures.

MDA-MB-231 cells were grown in McCoy's 5A Modified Media (Gibco) supplemented with 10% FBS (Hyclone). Py8119 cells were grown in F12K nutrient media (Gibco) supplemented with 5% Fetal Clone II (Fisher Scientific). MLO-Y4 cells were cultured on rat-tail collagen type I (BD Biosciences) coated cell culture plates. Cells were cultured in α-modified essential medium (α-MEM) (Gibco) supplemented with 2.5% FBS and 2.5% bovine calf serum (BCS) (Hyclone). All cell lines were incubated in a 5% CO₂ incubator at 37° C.

Conditioned Media (CM) Preparation.

MLO-Y4 cells were seeded onto 150 mm dishes (Corning) and incubated for 24 hr to allow attachment, after which media was removed and changed with α-modified essential medium (α-MEM) without phenol red (Gibco) supplemented with 2.5% FBS and 2.5% BCS (Hyclone). MLO-Y4 cells were incubated in the absence or present of 20 μM AD in a 5% CO2 incubator at 37° C. for 48 hr and the CM was collected.

Cell Proliferation Assay.

Cell viability was assessed using WST-1 (Water Soluble Tetrazolium salts) assay (Roche). A single cell suspension was plated in 96-well plates at 2.0×10⁴ cells/well and allowed to attach to the plates at 37° C. for 2 hr. The cells were then treated with CM collected from MLO-Y4 cells treated with or without 20 μM AD for 18 h. After the treatment, cell viability was measured by adding 10 μl of Cell Proliferation Reagent WST-1 to each well and incubated for 1 hr at 37° C. in a 5% CO₂ incubator. The cell proliferation was measured at an emission wavelength of 450 nm with a Synergy HT Multi-Mode Microplate Reader (Biotek).

Cell Migration Assay.

Migration assays were performed in transwell membrane filter inserts in 24-well tissue culture plates (BD Biosciences San Jose, Calif., USA). The transwell membrane filter inserts contained 6.5-mm diameter, 8-μm pore size, 10-nm thick polycarbonate membranes. The breast cancer cell lines were harvested and resuspended in CM from MLO-Y4 cells with or without other compounds. Five-hundred microliter breast cancer cell suspensions were added to the upper side of the inserts at a density of 10×10⁴ cells/insert and 750 μl CM with or without other compounds was added to the lower wells. Cells were incubated at 37° C. for 18-20 hr. Cells that did not migrate through the filters were removed using cotton swabs, and cells that migrated through the inserts were fixed and stained with Hema 3 Stat Pack (Fisher Scientific). The number of migrated cells in 5 fields of view per insert was counted under a light microscope at magnification 10×.

Soft Agar Colony Formation Assay.

For anchorage-independent cell growth, MDA-MB-231 cells were plated in 0.4% agarose with complete medium supplemented with either 100 μM ATPγS or 200 μM adenosine on top of a 0.8% agarose base supplemented with complete medium. Cells were maintained for about 2 weeks before staining with p-iodonitrotetrazolium violet (Sigma-Aldrich, St. Louis, Mo.). Images were captured by using a scanner and the numbers of colonies were counted.

Animals.

Four-week-old female athymic nude mice (Harlan Sprague-Dawley, Indianapolis, Ind., USA) were used for the mammary fat pad injections. Four- to five-week old female C57b1/6 mice were used for the intratibial injections. Animals were maintained under the care and supervision of the Laboratory Animal Research facility at the University of Texas Health Science Center, San Antonio, Tex. The animal protocol was approved and monitored by the Institutional Animal Care and Use Committee.

In Vivo Xenograft Experiment.

MDA-MB-231 cells were injected subcutaneously in the mammary fat pad of 4-week-old female nu/nu athymic nude mice. Each mouse received bilateral subcutaneous inoculation in both the left and right inguinal mammary fat pad areas with 100 μl of cell suspension containing ˜1×10⁷ cells/ml in serum-free media. Animals were randomly assigned to 3 different groups, and solid tumors were allowed to form up to about 5 mm³ volume before treatments began. ATPγS, at 400 μmol/500 μl saline, adenosine, at 400 μmol/500 μl saline, or 500 μl of saline, were administered intraperitoneally (IP) three times a week for 3 weeks. The growth of xenograft tumors was monitored twice a week and tumor size was measured with a caliper in two dimensions. Tumor volumes were calculated with the equation V=(L×W²)×0.5 (mm³), where L is length and W is width of a tumor.

Intratibial Injections.

Mice were anesthetized by isoflurane and were also given buprenorpine-HCl (0.3 mg/ml) as an analgesic. Py8119 cells expressing Luc-GFP (1×10⁵ in 20 μl of PBS) were inoculated into the bone marrow area of right tibias through the pre-made hole by a Hamilton syringe fitted with a 30-gauge needle. PBS was injected into the left tibias as control. ATPγS, at 400 μmol/500 μl saline or 500 μl of saline, was administered IP twice a week for 5 weeks, beginning from day 1. Intratibial tumor growth was monitored with bioluminescence imaging with a Xenogen IVIS-Spectrum imaging system (Xenogen, Alameda, Calif., USA) every week starting from 3 days after tumor cell inoculation.

Bioluminescence Imaging Analysis.

Mice were anesthetized and D-luciferin (Caliper Life Sciences, Alameda, Calif.) was injected IP at 75 mg/kg in PBS. Xenogen IVIS Spectrum Imaging system was used to acquire bioluminescence images at 10 min after injection. Acquisition time was set at 60 sec at the beginning and reduced later on in accordance with signal strength to avoid saturation. Analysis was performed using LivingImage software (Xenogen) by measurement of photon flux (measured in photons/sec/cm²/steradian) with a region of interest (ROI) drawn around the bioluminescence signal to be measured. Tumor burden was taken by drawing an ROI around the major bioluminescence signal from the hind limb.

Statistical Analysis.

Unless otherwise specified in the Figure Legends, the data are presented as the mean±S.E.M. of at least three determinations. Asterisks indicate the degree of significant differences compared with the controls (*, P<0.05; **, P<0.01; ***, P<0.001). One-way analysis of variance (ANOVA) and Student Newman-Keuls test were used to compare groups using GraphPad Prism 5.04 software (GraphPad).

1. A method for treating a cancer patient comprising administering to the patient an effective amount of a purinergic P2 receptor agonist.

2. The method of claim 1, wherein the purinergic P2 receptor agonist is a non-hydrolysable ATP analog.

3. The method of claim 2, wherein the non-hydrolysable ATP analog is adenosine 5′-[α-thio]triphosphate (ATPaS); alpha,beta-methylene-adenosine-5′-diphosphate (ApCpp); beta,gamma-methylene-ATP (AppCp); adenosine 5′-[γ-thio]triphosphate (ATPγS); adenylyl imidodiphosphate (AMP-PNP); N⁶-diethyl-beta,gamma-dibromomethylene-ATP; 2-methylthio-ATP (APM); alpha,beta-methylene-ATP; beta,gamma-methylene-ATP; di-adenosine pentaphosphate (Ap5A); 1,N⁶-ethenoadenosine triphosphate; adenosine 1-oxide triphosphate; 2′,3′-O-(benzoyl-4-benzoyl)-ATP (B-ZATP); or 2′,3′-O-(2,4,6-trinitrophenyl)-ATP (TNP-ATP).

4. The method of claim 2, wherein the non-hydrolysable ATP analog is ATP-γ-S.

5. The method of claim 1, wherein the purinergic P2 receptor agonist further comprises a targeting agent.

6. The method of claim 5, wherein the targeting agent is a cancer cell specific ligand.

7. The method of claim 1, wherein the cancer is breast cancer.

8. The method of claim 1, wherein the purinergic P2 receptor agonist is administered by local injection.

9. The method of claim 1, wherein the purinergic P2 receptor agonist is administered by systemically.

10. The method of claim 1, further comprising administering an adenosine receptor antagonist.

11. The method of claim 10, wherein the adenosine receptor antagonist is N-(4-cyanophenyl)-2-[4-(2,3,6,7-tetrahydro-2,6-dioxo-1,3-dipropyl-1H-purin-8-yl)phenoxy]-acetamide (MRS 1754); N-(4-acetylphenyl)-2-[4-(2,3,6,7-tetrahydro-2,6-dioxo-1,3-dipropyl-1H-purin-8-yl)phenoxy]acetamide (MRS 1706); 8-[4-[4-(4-Chlorobenzyl)piperazide-1-sulfonyl)phenyl]]-1-propylxanthine (PSB 0788); 4-(2,3,6,7-Tetrahydro-2,6-dioxo-1-propyl-1H-purin-8-yl)-benzenesulfonic acid (PSB 1115); 8-Ethoxy-9-ethyl-9H-purin-6-amine (ANR94); or 8-[4-[4-(4-Chlorophenzyl)piperazide-1-sulfonyl)phenyl]]-1-propylxanthine (PSB 603).

12. The method of claim 10, wherein the adenosine receptor antagonist is MRS 1754.

13. The method of claim 10, wherein the adenosine receptor antagonist is ANR94

14. The method of claim 10, wherein the purinergic P2 receptor agonist and adenosine receptor antagonist are administered within 1, 5, 10, 20, 30, or 60 minutes of each other.

15. The method of claim 10, wherein the purinergic P2 receptor agonist and adenosine receptor antagonist are administered concurrently.

16. The method of claim 1, wherein the cancer is a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterine cancer.

17. The method of claim 16, wherein the cancer is a lung, breast, or prostate cancer.

18. The method of claim 16, wherein the cancer is a metastatic cancer.

19. A method for treating a cancer patient comprising administering to the patient an effective amount of an adenosine receptor antagonist.

20. The method of claim 20, wherein the adenosine receptor antagonist is N-(4-cyanophenyl)-2-[4-(2,3,6,7-tetrahydro-2,6-dioxo-1,3-dipropyl-1H-purin-8-yl)phenoxy]-acetamide (MRS 1754); N-(4-acetylphenyl)-2-[4-(2,3,6,7-tetrahydro-2,6-dioxo-1,3-dipropyl-1H-purin-8-yl)phenoxy]acetamide (MRS 1706); 8-[4-[4-(4-Chlorobenzyl)piperazide-1-sulfonyl)phenyl]]-1-propylxanthine (PSB 0788); 4-(2,3,6,7-Tetrahydro-2,6-dioxo-1-propyl-1H-purin-8-yl)-benzenesulfonic acid (PSB 1115); 8-Ethoxy-9-ethyl-9H-purin-6-amine (ANR94); or 8-[4-[4-(4-Chlorophenzyl)piperazide-1-sulfonyl)phenyl]]-1-propylxanthine (PSB 603).

21. The method of claim 19, wherein the adenosine receptor antagonist is 8-[4-[((4-cyanophenyl)carbamoylmethyl)oxy]phenyl]-1,3-di(n-propyl)xanthine hydrate (MRS 1754).

22. The method of claim 19, wherein the adenosine receptor antagonist is ANR94.

23. The method of claim 19, wherein the adenosine receptor is an A2B adenosine receptor.

24. The method of claim 19, wherein the adenosine receptor is an A2A adenosine receptor.

25. The method of claim 19, wherein the adenosine receptor antagonist is administered by local injection.

26. The method of claim 25, wherein the local injection is an intratumoral injection.

27. The method of claim 19, wherein the cancer is a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterine cancer.

28. The method of claim 27, wherein the cancer is a lung, breast, or prostate cancer.

29. The method of claim 27, wherein the cancer is a metastatic cancer.
